SEQUENCE LISTING (17 sequences)

SEQ ID NO: 1 - DNA, 41 bp, Hop latent viroid
  source 1..41: mol_type = genomic DNA
gaacaagaagaagccgaagcaacgaaacctactcgagcgag

SEQ ID NO: 2 - DNA, 41 bp, Hop latent viroid; identical to SEQ ID NO: 1
  source 1..41: mol_type = genomic DNA

SEQ ID NO: 3 - DNA, 40 bp, Hop latent viroid
  source 1..40: mol_type = genomic DNA
aacggctccttcttcacaccctcaagagttgtatccaccg

SEQ ID NO: 4 - DNA, 20 bp, Hop latent viroid
  source 1..20: mol_type = genomic DNA
cgtgacttacctgtatggtg

SEQ ID NO: 5 - DNA, 20 bp, Hop latent viroid
  source 1..20: mol_type = genomic DNA
tgaacttctgcaggtaaagc

SEQ ID NO: 6 - DNA, 18 bp, Hop latent viroid
  source 1..18: mol_type = genomic DNA
gaactggcgctcgatctc

SEQ ID NO: 7 - DNA, 19 bp, Hop latent viroid
  source 1..19: mol_type = genomic DNA
agccggagttggaaactac

SEQ ID NO: 8 - DNA, 13 bp, Hop latent viroid
  source 1..13: mol_type = genomic DNA
cttcttcttgttc

SEQ ID NO: 9 - DNA, 13 bp, Hop latent viroid
  source 1..13: mol_type = genomic DNA
cttcttattgttc

SEQ ID NO: 10 - DNA, 37 bp, Cannabis sativa
  source 1..37: mol_type = genomic DNA
gccgcatcagcttgtgttgacgttgttgtgcttgact

SEQ ID NO: 11 - DNA, 37 bp, Cannabis sativa
  source 1..37: mol_type = genomic DNA
tgaagcaggcatggacaccagcttctgatgagttgcg

SEQ ID NO: 12 - DNA, 18 bp, Cannabis sativa
  source 1..18: mol_type = genomic DNA
atcactatgacggtggct

SEQ ID NO: 13 - DNA, 20 bp, Cannabis sativa
  source 1..20: mol_type = genomic DNA
atccatcttgttcacagcaa

SEQ ID NO: 14 - DNA, 21 bp, Cannabis sativa
  source 1..21: mol_type = genomic DNA
ccagagatcatgttgggaaca

SEQ ID NO: 15 - DNA, 20 bp, Cannabis sativa
  source 1..20: mol_type = genomic DNA
tgaagggacaaacacgagag

SEQ ID NO: 16 - DNA, 10 bp, Cannabis sativa
  source 1..10: mol_type = genomic DNA
ctgatgcggc

SEQ ID NO: 17 - DNA, 10 bp, Cannabis sativa
  source 1..10: mol_type = genomic DNA
ctgatgcagc

(19) United States
(12) Patent Application Publication
(10) Pub. No.: US 2024/0410025 A1
(43) Pub. Date: Dec. 12, 2024

(54) METHOD FOR RAPIDLY AND ACCURATELY DETECTING HOP LATENT VIROID RNA

(71) Applicant: Colorado Genomics LLC, Fort Collins, CO (US)

(72) Inventors: Tassa K. Saldi, Erie, CO (US); Erika L. Lasda, Westminster, CO (US); Alfonso Garrido-Lecca, Erie, CO (US); Patrick K. Gonzales, Fort Collins, CO (US)

(73) Assignee: Colorado Genomics LLC, Fort Collins, CO (US)

(21) Appl. No.: 18/737,026

(22) Filed: Jun. 7, 2024

Related U.S. Application Data
(60) Provisional application No. 63/627,640, filed on Jan. 31, 2024.
(60) Provisional application No. 63/507,395, filed on Jun. 9, 2023.

(51) Int. Cl. C12Q 1/70; C12N 15/10; C12Q 1/37; C12Q 1/48; C12Q 1/6806; G01N 21/64

(52) U.S. Cl. CPC C12Q 1/70; C12N 15/1096; C12Q 1/37; C12Q 1/485; C12Q 1/6806; G01N 21/6456; G01N 21/6486; G01N 2333/9126; G01N 2333/9128; G01N 2333/924

(57) ABSTRACT

Methods for rapidly and accurately detecting genetic material from hop latent viroid RNA (HLVd) that combines reverse transcription loop-mediated isothermal amplification (RT-LAMP) technology with specific oligonucleotide primers, fluorophore-labeled oligonucleotide primers, quencher-conjugated oligonucleotide primers, pH buffers, and enzymes, are described. The methods include at least one internal positive control targeting sequence, for minimizing false positive and false negative results, thereby allowing more certain interpretation of the results. The reaction can be performed at a single elevated temperature, can be completed in 1-1.5 hours, and the results can readily be interpreted by visually observing the fluorescence color of the reaction using ultraviolet light, or by using an electronic image acquisition system for viewing fluorescence results from one or more reactions.

### INCORPORATION BY REFERENCE OF THE SEQUENCE LISTINGS

The accompanying XML Sequence Listing named <ST26SequenceListing dtdVersion=“V1_3” fileName=“TUMI.02USU1GeneSequence17. xml” softwareName=“WIPO Sequence” softwareVersion=“2.3.0” productionDate=“2024-06-07”>Size=16.0 KB) submitted on Jun. 7, 2024 in U.S. patent application Ser. No. 18/737,026 is hereby incorporated by reference.

### BACKGROUND

Hop latent viroid (HLVd) is a pathogenic circular RNA that infects plants including Cannabis plants. HLVd infects the plant systemically, can be asymptomatic until later in the plant life cycle and can cause a huge loss in crop yield. Early detection of infection can help save crops and resources and prevent infection spread by identifying and removing infected plants.

HLVd can remain latent in a Cannabis plant for long periods before exhibiting signs such as irregular branching, decreased trichome production, chlorosis of the leaves, and stunted development. Asymptomatic plants appear healthy, but are actively transmitting the viroid to the remainder of the crop.

This viroid spreads efficiently from plant to plant through mechanical transmission, which means that a contaminated plant must come into direct or indirect contact with a healthy plant, or by pruning an infected plant and then pruning an uninfected plant with tools such as scissors, scalpels, shears, as examples. The viroid can also be spread through water runoff and within the seeds of a diseased mother plant.

### SUMMARY

Embodiments of the present invention include a method for rapidly and accurately detecting target nucleic acid, having the following steps: (1) collecting a sample of the plant to be investigated; (2) transferring a small amount of the sample to an optically clear reaction test tube containing: (a) a reverse transcriptase (an enzyme used to generate complementary DNA (cDNA) from an RNA template); (b) deoxyribonucleotide triphosphates (dNTPs) (the building blocks of DNA, which lose two of phosphate groups when incorporated into DNA during replication); (c) a strand-displacement DNA polymerase (an enzyme that catalyzes the synthesis of DNA from nucleoside triphosphates, by adding nucleotides to the (3′)-end of a DNA strand, one nucleotide at a time; (d) plant control nucleic acid RNA introduced as part of the sample in step (2) as a plant transcript; (e) oligonucleotide primers specifically contacting the target nucleic acid sequences of either the pathogen nucleic acid or the plant control nucleic acid; (f) fluorophore-conjugated oligonucleotide primers for specifically contacting the target nucleic acid sequences of either the pathogen nucleic acid or the plant control gene nucleic acid; and (g) quencher-conjugated oligonucleotides specifically contacting the fluorophore-conjugated oligonucleotide primers, the requisite materials for a reverse transcription loop-mediated isothermal amplification reaction (RT-LAMP) (It should be mentioned that the reactions may be performed in optically clear microplates containing a chosen number of individual wells.); (3) incubating the reaction under conditions such that cDNA synthesis and isothermal amplification takes place, thereby generating DNA amplification products; (4) cooling the resulting reaction products under conditions permissive for oligonucleotide hybridization thereby quenching unincorporated fluorophore-conjugated oligonucleotide primers; (5) detecting specific signals for incorporated fluorophore-conjugated oligonucleotide primers, wherein detection of one fluorophore signal indicates the presence of the plant control nucleic acid and correct functioning of the reaction, and detection of the other fluorophore signal indicates the presence of the pathogen nucleic acid; and (6) interpretating the results, wherein detection of either or both fluorophore signals indicates the presence of the target nucleic acids present in the sample, while failure to detect the pathogen fluorophore signal indicates the absence of target nucleic acid in the sample, and failure to detect any fluorophore signal indicates that the reaction has failed.

### BRIEF DESCRIPTION OF THE DRAWINGS

The accompanying drawings, which are incorporated in and form a part of the specification, illustrate the embodiments of the present invention and, together with the description, serve to explain the principles of the invention. In the drawings:

FIG. 1 is a flow diagram of an embodiment of the present method.

FIG. 2A(1) illustrates the hybridization of the reverse complimentary sequence for the HLVd Forward Internal Primer, FIP, conjugated at the 3′ end to a dark quencher, BHQ2, for the TexasRed fluorophore of SEQ ID NO: 8, with the FIP primer conjugated to the TexasRed fluorophore of SEQ ID NO: 2, and FIG. 2A(2) illustrates the hybridization of the reverse complimentary sequence for the HLVd Forward Internal Primer, FIP, conjugated at the 3′ end to a dark quencher, BHQ2, for the TexasRed fluorophore of SEQ ID NO: 9, with the FIP primer conjugated to the TexasRed fluorophore of SEQ ID NO: 2, showing the mismatch marked by the arrow, of an A (adenine) nucleotide bound to a G (guanine) nucleotide instead of a C (cytosine) nucleotide bound to the G (guanine) nucleotide; while FIG. 2B(1) illustrates the hybridization of the reverse complimentary sequence for the Cannabis Forward Internal Primer, FIP, conjugated at the 3′ end to a dark quencher, BHQ1, for the FAM fluorophore of SEQ ID NO: 16, with the FIP primer having SEQ ID NO: 10 conjugated to the FAM fluorophore, and FIG. 2B(2) illustrates the hybridization of the reverse complimentary sequence for the Cannabis Forward Internal Primer, FIP, conjugated at the 3′ end to a dark quencher, BHQ1, for the FAM fluorophore of SEQ ID NO: 17, with the FIP primer having SEQ ID NO: 10 conjugated to the FAM fluorophore, showing the mismatch marked by the arrow, of an A (adenine) nucleotide bound to a C (cytosine) nucleotide instead of a G (guanine) nucleotide bound to the C nucleotide.

FIG. 3 is a schematic representation of an embodiment of the electronic image acquisition system of the present invention for viewing fluorescence results from one or more reactions.

FIG. 4 shows algorithm for analyzing images taken by the camera shown in FIG. 3 hereof, which captures an image of the reaction tube holder showing fluorescence from the reaction tubes.

### BRIEF DESCRIPTION OF THE SEQUENCE LISTINGS

SEQ ID NO: 1 discloses the (5′ to 3′) nucleic acid sequence for the HLVd Forward Internal Primer, FIP.

SEQ ID NO: 2 discloses the (5′ to 3′) nucleic acid sequence for the HLVd Forward Internal Primer, FIP, conjugated to a fluorophore at the 5′ end.

SEQ ID NO: 3 discloses the (5′ to 3′) nucleic acid sequence for the HLVd Backward Internal Primer, BIP.

SEQ ID NO: 4 discloses the (5′ to 3′) nucleic acid sequence for the HLVd Forward External Primer, F3.

SEQ ID NO: 5 discloses the (5′ to 3′) nucleic acid sequence for the HLVd Backward External Primer, B3.

SEQ ID NO: 6 discloses the (5′ to 3′) nucleic acid sequence for the HLVd Forward Loop Primer, Loop F.

SEQ ID NO: 7 discloses the (5′ to 3′) nucleic acid sequence for the HLVd Backward Loop Primer, Loop B.

SEQ ID NO: 8 discloses the (5′ to 3′) nucleic acid reverse complimentary sequence for the HLVd Forward Internal Primer, FIP, conjugated at the 3′ end to a dark quencher for the fluorophore identified in SEQ ID NO: 2.

SEQ ID NO: 9 discloses an alternate (5′ to 3′) nucleic acid reverse complimentary sequence for the HLVd Forward Internal Primer, FIP, conjugated at the 3′-end to a dark quencher for the fluorophore identified in SEQ ID NO: 2.

SEQ ID NO: 10 discloses the (5′ to 3′) nucleic acid sequence for the Cannabis Forward Internal Primer, FIP, conjugated to a fluorophore at the 5′ end.

SEQ ID NO: 11 discloses the (5′ to 3′) nucleic acid sequence for the Cannabis Backward Internal Primer, BIP.

SEQ ID NO: 12 discloses the (5′ to 3′) nucleic acid sequence for the Cannabis Forward External Primer, F3.

SEQ ID NO: 1304 discloses the (5′ to 3′) nucleic acid sequence for the Cannabis Backward External Primer, B3.

SEQ ID NO: 14 discloses the (5′ to 3′) nucleic acid sequence for the Cannabis Forward Loop Primer, Loop F.

SEQ ID. NO: 15 discloses the (5′ to 3′) nucleic acid sequence for the Cannabis Backward Loop Primer, Loop B.

SEQ ID NO: 16 discloses a (5′ to 3′) nucleic acid reverse complimentary sequence for the Cannabis Forward Internal Primer, FIP, conjugated at the 3′ end to a dark quencher for the fluorophore identified in SEQ ID NO: 10.

SEQ ID NO: 17 discloses an additional (5′ to 3′) nucleic acid reverse complimentary sequence for the Cannabis Forward Internal Primer, FIP, conjugated at the 3′ end to a dark quencher for the fluorophore identified in SEQ ID NO: 10.

### DETAILED DESCRIPTION

It is desirable to have a process for detection of target genetic material that uses low-complexity, non-invasive biological sample collection techniques, that is rapid, easy-to-use, highly accurate, does not require specialized laboratory equipment, as most current viral/subviral molecular tests rely on expensive temperature-cycling machines that are not available in most settings, and that does not require significant specialized training to collect a sample, perform the reaction, or interpret the results. Embodiments of the present method combine reverse transcription, loop-mediated isothermal amplification (RT-LAMP) technology with oligonucleotide primers, fluorophore-labeled oligonucleotides, quencher technology, buffer components, enzymes, and enzyme ratios, chosen to provide a high level of sensitivity and to minimize the false positive and false negative results that often accompany the use of RT-LAMP. The present method includes positive control targeting sequences, thereby allowing significant confidence in the interpretation of results, can be performed at a single elevated temperature, and can be completed in 1-2 h. Further, the results of the reactions can readily be interpreted by observing the fluorescence color of the reaction using ultraviolet light.

Loop-mediated isothermal amplification, LAMP, technology has been used to detect pathogens, such as malaria and salmonella, as examples. LAMP merged with reverse transcriptase, RT-LAMP, has been used to detect viral RNA in HIV and several respiratory RNA viruses, including SARS-CoV-2. Hairpin-forming LAMP primers first invade the DNA template, which is then annealed and extended as catalyzed by a strand-displacing DNA polymerase. In the initiation of amplification, the annealed primers are used to prime the action of a strand displacement enzyme, leading to the formation of a dumbbell-like single-strand DNA loops, which form the basis for amplification and elongation. Forward and backward inner LAMP primers hybridize to the complementary and reverse complimentary target sequences. The product of LAMP is a series of concatemers of the target region.

Because LAMP uses 4-6 primers targeting 6-8 regions within a small segment of the genome, which primers have many constraints, commercial software is often used to assist with primer design. The large number of primers per target increase the likelihood of primer-primer interaction, and the incidence of false positive and false negative results. See, “Reduced False Positives and Improved Reporting of Loop-Mediated Isothermal Amplification Using Quenched Fluorescent Primers” by Patrick Hardinge and James A. H. Murray, in Scientific Reports 9, Article Number 7400 (2019). As will be discussed in detail below, careful primer design and optimal reaction conditions and testing are used to minimize these problems.

Briefly, embodiments of the present invention include detecting the presence of hop latent viroid (HLVd) infection in Cannabis plants (by targeting HLVd nucleic acid/RNA/genetic material), in addition to detecting the presence of a control plant gene using two-color fluorescence RT-LAMP technology. An example of a useful control plant gene target is the ubiquitously expressed Cannabis plant gene nucleic acid (Cannabis sativa EF-1-alpha gene sequence nucleic acid is the transcript target amplified for the control; however, any sequence from the genome could be used)/RNA/genetic material. A small amount of plant material is added to a lysis/stabilization solution in a first tube. As stated above, the Cannabis control plant gene target is already expressed in the plant material and is a transcript targeted by the present method. A small volume of the liquid lysate from this tube is transferred to a reaction tube containing reagents necessary for the RT-LAMP enzymatic process, and fluorescent detection.

Reagents include enzymes, primer oligonucleotides, fluorophore-conjugated oligonucleotides, quencher oligonucleotides, buffer components, and other chemicals/components for reducing false positives, reducing background (template) fluorescence, increasing positive fluorescence, increasing assay sensitivity/accuracy, and increasing visual differences. If plant material is detected, the resulting materials will fluoresce green under UV light. If HLVd material is detected, the resulting materials will fluoresce red under UV light. If the assay has failed, or there is insufficient sample quantity or quality, the resulting materials will not fluoresce.

WIPO Publication No. WO 2022/133137 for “Method For Rapidly And Accurately Detecting SARS-COV-2 Nucleic Acid,”, was published on 23 Jun. 2022, but differs from embodiments of the present invention, among other features, in the specific oligonucleotides for both HLVd and Cannabis as opposed to SARS-COV-2 and human saliva; the lysis/stabilization solution, and the buffers and other chemical components of the reaction.

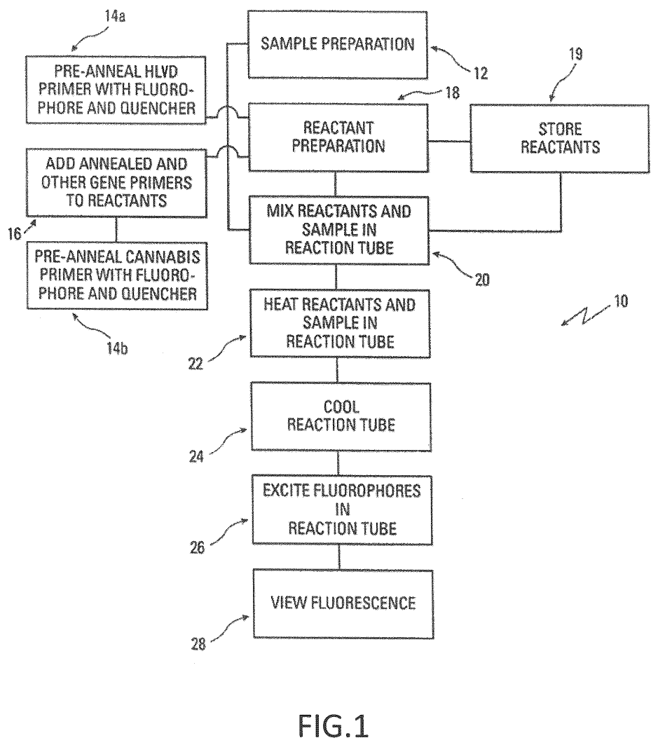

Reference will now be made in detail to the present embodiments of the invention, examples of which are illustrated in the accompanying drawings. It will be understood that the FIGURES are presented for the purpose of describing particular embodiments of the invention and are not intended to limit the invention thereto. Turning now to FIG. 1, represented is a flow diagram, 10, of an embodiment of the present method. In step 12, Sample (Plant Lysate) Preparation, a small amount of plant material (typically 0.12 g to 0.6 g of root tissue, or 0.06 g to 0.12 g of leaf tissue) from the plant to be analyzed for HLVd is collected, and placed in a 5 mL microcentrifuge collection tube having a lid and containing about 3.5 mL of 10 mM Tris-HCl buffer (pH 8), for stabilizing the pH of the solution, 0.1 mM EDTA (Ethylenediaminetetraacetic acid, for disrupting the outer membrane of cells by chelating Mg⁺² ions, essential to outer membrane stability), 75 mM Trehalose, 0.2 mg/mL BSA (Bovine Serum Albumin), 0.2% Tween-20 (percentage dilution from the concentrate, to assist in overcoming the effects of PCR inhibitors inherent in plant tissue), 0.2 g of activated charcoal, 0.05 g of Chelex 100 (50-100 mesh) Resin (a styrene divinylbenzene copolymer for stabilizing the DNA/RNA in the solution), and 2, 4.5 mm glass beads. The test tube is then capped and shaken vigorously for about 10 s, forming a lysate. The shaken tube is then shaken in e downward direction to cause the liquid to move to the bottom of the collection tube, and placed in a rack for about 5 min. to permit impurities to settle to the bottom.

TABLE 1 is a list of the HLVd and Cannabis primers used in the reactant mixture.

TABLE 1

| | | Primary | | |
|---|---|---|---|---|
| SEQ ID | | or | Primer | |
| NO: | Target | Alternate | Name | Sequence (5′ to 3′) |
| 1 | HLVd | Primary | FIP | GAA CAA GAA GAA GCC GAA GCA ACG |
| | | | | AAA CCT ACT CGA GCG AG |
| 2 | HLVd | Primary | FIP label | TexasRed-GAA CAA GAA GAA GCC GAA |
| | | | | GCA ACG AAA CCT ACT CGA GCG AG |
| 3 | HLVd | Primary | BIP | AAC GGC TCC TTC TTC ACA CCC TCA |
| | | | | AGA GTT GTA TCC ACC G |
| 4 | HLVd | Primary | F3 | CGT GAC TTA CCT GTA TGG TG |
| 5 | HLVd | Primary | B3 | TGA ACT TCT GCA GGT AAA GC |
| 6 | HLVd | Primary | LF | GAA CTG GCG CTC GAT CTC |
| 7. | HLVd | Primary | LB | AGC CGG AGT TGG AAA CTA C |
| 8 | HLVd | Primary | Quench1 | CTT CTT CTT GTT C-BHQ2 |
| 9 | HLVd | Alternate | Quench2 | CTT CTT ATT GTT C-BHQ2 |
| 10 | Cannabis | Primary | FIP label | FAM-GCC GCA TCA GCT TGT GTT GAC |
| | | | | GTT GTT GTG CTT GAC T |
| 11 | Cannabis | Primary | BIP | TGA AGC AGG CAT GGA CAC CAG CTT |
| | | | | CTG ATG AGT TGC G |
| 12 | Cannabis | Primary | F3 | ATC ACT ATG ACG GTG GCT |
| 13 | Cannabis | Primary | B3 | ATC CAT CTT GTT CAC AGC AA |
| 14 | Cannabis | Primary | LF | CCA GAG ATC ATG TTG GGA ACA |
| 15 | Cannabis | Primary | LB | TGA AGG GAC AAA CAC GAG AG |
| 16 | Cannabis | Primary | Quench1 | CTG ATG CGG C-BHQ1 |
| 17 | Cannabis | Primary | Quench2 | CTG ATG CAG C-BHQ1 |

Step 14a of FIG. 1 illustrates the pre-annealing of a fluorophore with a quencher for detection of HLVd. The FIP primer having SEQ ID NO: 1 was chosen for conjugation to a Texas Red or another fluorophore at the 5′ end, generating SEQ ID NO: 2 (FIP-TR). It should be mentioned that one of the other oligonucleotide primers, BIP, F3, B3, Loop F, and Loop B, could have been selected in place of the FIP primer. The quencher used for targeting the Texas Red fluorophore is BHQ2 and the oligonucleotide sequence having SEQ ID NO: 8 (Quencher1) is reverse complementary to the oligonucleotide conjugated to the Texas Red fluorophore with the BHQ2 located at the 3′ end.

The primers and quenchers were supplied in separate tubes as lyophilized (freeze dried) components from a supplier thereof, with the fluorophore and the quencher conjugated to their respective oligonucleotides as purchased, and are then independently dissolved in water to a concentration of 100 μM. Primer and quencher aqueous solutions were mixed together at an experimentally determined ratio of 1:2 of primer (SEQ ID NO: 2) to quencher (SEQ ID NO: 8). Other ratios that may yield usable results can range from 1:1 to 1:2.5 of primer to quencher. This mixture was heated to about 85° C. for about 2 min. to remove any secondary structure of the oligonucleotides, and slowly cooled to room temperature. Slow cooling allows the quencher to bind or anneal to the primer. Step 14b illustrates the pre-annealing step for the Cannabis primer SEQ ID NO: 10 (FIP-FAM) and quencher primer SEQ ID NO: 16 (Quencher1) in a different tube in a 1:1 ratio, and was identically and separately performed from the pre-annealing of the FIP/Quencher combination for the HLVd FIP and quencher set forth above in Step 14a, and forming Mixture 1. Other ratios that may yield usable results can range from 1:1 to 1:2.5 of primer to quencher. A second mixture, Mixture 2, was pre-annealed in another tube using SEQ ID NO: 10 (FIP-FAM) and SEQ ID NO: 17 (Quencher2) in a ratio 1:1.5 of FIP-FAM to Quencher2. Other ratios that may yield usable results can range from 1:1 to 1:2.5. Both Mixture 1 and Mixture 2 were separately heated to about 85° C. for about 2 min. to remove any secondary structure of the oligonucleotides, and slowly cooled to room temperature. The FIP primer having SEQ ID NO: 10 was chosen for conjugation to a FAM or another fluorophore, and the oligonucleotide sequences having SEQ ID NO: 16 and SEQ ID NO: 17 were conjugated to a BHQ1 quencher, and are reverse complementary to the oligonucleotide conjugated to the FAM fluorophore. It should be mentioned that one of the other oligonucleotide primers, BIP, F3, B3, Loop F, and Loop B, could have been selected in place of the FIP primer. The resulting pre-annealed solution from Step 14a and both Mixture 1 and Mixture 2 from Step 14b were then mixed with the remaining primers (BIP, F3, B3, Loop F, and Loop B) and added to the reaction mixture in the concentrations specified in TABLES 2 and 3, as part of Step 16, which increases the stability of the premixed reagents.

It should be mentioned that the alternate primer, SEQ ID NO 9 was selected as a primer that could work in the situation where the primary primer, SEQ ID NO 8 failed to provide useful results. However, it was found that both primary and alternate primers generated similar results.

TABLE 2 illustrates the HLVd primer solution containing 27.5 times the concentrations generally used for analyses.

TABLE 2

| SEQ ID | Primer | Final |
|---|---|---|
| NO: | Name | Concentration |
| 4 | F3 | 3.3 μM |
| 5 | B3 | 3.3 μM |
| 6 | LF | 6.6 μM |
| 7 | LB | 6.6 μM |
| 3 | BIP | 26.4 μM |
| 1 | FIP | 13.2 μM |
| | (unlabeled) | |
| 2 | FIP-TR | 13.3 μM |
| 8 | Quench | 26.6 μM |

Note
that SEQ ID NO: 2 and SEQ ID NO: 8 have been pre-annealed in Step 14a.

TABLE 3 illustrates the Cannabis primer solution containing 33 times the concentrations generally used for analyses. The 33× and 27.5× concentrations are formulated to make the final volume of each reaction as small as possible, which is necessary for lyophilization. Those concentrations are the highest that are available using a 100 μM stock solution.

TABLE 3

| SEQ ID | Primer | Final |
|---|---|---|
| NO: | Name | Concentration |
| 12 | F3 | 3.3 μM |
| 13 | B3 | 3.3 μM |
| 14 | LF | 6.6 μM |
| 15 | LB | 6.6 μM |
| 11 | BIP | 14.52 μM |
| 10 | FIP-FAM (Mix 1) | 24.6 μM |
| 16 | Quench 1 (Mix 1) | 24.6 μM |
| 10 | FIP-FAM (Mix 2) | 6.56 μM |
| 17 | Quench 2 (Mix 2) | 9.84 μM |

Note
that SEQ ID NO: 10 and SEQ ID NO: 16 have been pre-annealed in Step 14b as Mixture 1, and SEQ ID NO: 10 and SEQ ID NO: 17 have been pre-annealed in Step 14b as Mixture 2. It should be mentioned that any target control gene nucleic acid molecule can be utilized.

Analysis (reactant) solutions are prepared in Step 18 by combining the components set forth in TABLE 4.

TABLE 4

| Reagent | Final Concentration |
|---|---|
| Tris-HCl | 20 mM |
| Ammonium sulfate ((NH4)2SO4) | 10 mM |
| Potassium chloride (KCl) | 50 mM |
| Magnesium sulfate (MgSO4) | 8 mM |
| Tween ® 20 | 0.1% |
| Deoxyguanosine triphosphate (dGTP) | 1.4 mM |
| Deoxycytidine triphosphate (dCTP) | 1.4 mM |
| Deoxyadenosine triphosphate (dATP) | 1.4 mM |
| Deoxythymidine triphosphate (dTTP) | 0.7 mM |
| Deoxyuridine triphosphate (dUTP) | 0.7 mM |
| NEB hotstart BST2.0 | 26.4 U |
| NEB warmstart RTx | 24 U |
| NEB Uracil-DNA Glycosylase (UDG) | 1.6 U |
| Betaine | 0.8 mM |
| HLVd Mixture from TABLE 2 | 0.75X |
| Cannabis Mixture from TABLE 3 | 0.5X |

Note
that U represents units of enzyme activity. Additionally, as an example, TABLE 2 contains 27.5X of the component concentrations, which is 27.5 times the concentrations thereof in a solution generally used for analysis (1X); therefore, 0.75X contains ¾ of the concentrations of HLVd primers of a 1X solution thereof, and 0.5X contains ½ of the concentrations of a 1X solution of Cannabis primers (TABLE 3) of a 1X solution thereof.

The solution of components in TABLE 4 may be lyophilized in 0.1 μL strip tubes forming a pellet in each tube for storage for later use in Step 19.

The listed components perform the following functions:
(a) Tris hydrochloride is a buffer that maintains the pH of that solution for allowing optimal activity of the enzymes.
(b) Ammonium sulfate and potassium chloride stabilize the enzymes that catalyze the reaction.
(c) Magnesium sulfate augments for catalytic activity of enzymes in the assay and adjusting its concentration allows increased or decreased reaction temperature.
(d) Tween 20 is a polysorbate-type nonionic surfactant, and assists in increasing the specificity of the reaction. Alternatives include Triton X and NP-40.
(e) dNTPs are free deoxy-triphosphates (A, T, C, and G) that serve as the raw materials for amplification. dNTPs from any source in either powdered or liquid form can be used.
(f) deoxyuridine triphosphate (dUTP) when used in combination with thermolabile uracil DNA glycosylase is a component that destroys carry-over contamination prior to new amplification, and minimizes false positives. This component is optional, but improves performance of the assay and also reduces the risk of cross-contaminating future assays with previously amplified results. Amplification products from one run of a test contaminating future reactions can lead to false positives and is a problem for any nucleic acid test, including both LAMP and PCR. If a small amount of amplification product contaminates a new test, that test will appear positive because the primers/enzymes will amplify that molecule regardless of the presence or absence of target viral nucleic acid. Including dUTP and thermolabile UDG (uracil DNA glycosylase; NEB catalog #M0372S (Antarctic Thermolabile UDG)), while not necessary for the reaction to proceed, reduces the chance of these false positives, since when included, dUTP is incorporated at some low frequency in the amplification products. Antarctic thermolabile uracil DNA glycosylase is an enzyme that degrades DNA molecules that contain dUTP preventing carry-over contamination from previous reactions, as described above. Any commercially available or lab produced enzyme that cleave DNA at sites of dUTP incorporation can be used in the assay. This component is optional, but improves performance and specificity of the assay, and reduces the risk of cross-contaminating future assays with previously amplified results. If those amplification products accidentally contaminate a new test (as happens regularly), the UDG enzyme in the new test degrades any nucleic acid containing UDG. However, this process only works at room temperature, and once the reaction is heated, the thermolabile UDG is inactivated and does not degrade the new amplicons of the new test.
(g) NEB hotstart BST 2.0 strand-displacement polymerase enzyme is an enzyme that catalyzes the amplification of the target DNA using added primer sets. Any strand displacement polymerase may be used including, but not limited to: BST 2.0, BST 2.0-hotstart, BST 3.0 (New England Biolabs), EquiPhi29 and Bsm DNA polymerases (ThermoFisher Scientific), losPol Bst (ArticZyme Technologies), or any in lab produced polymerase enzyme with strand displacement activity. Bst 2.0 DNA Polymerase (NEB catalog #M0537S) and/or Bst 2.0 WarmStart® DNA Polymerase (NEB catalog #M0538S) were employed.
(h) NEB WarmStart RTx (NEB catalog #M0380S WarmStart® RTx Reverse Transcriptase) is an enzyme that catalyzes the conversion of RNA into complementary DNA, allowing detection of RNA targets (such as RNA viruses and viroids). Any commercial or in-lab produced enzyme that converts RNA into complementary DNA may be used in the assay. This component is optional depending on the nature of the target to be amplified.
(i) Betaine (trimethylglycine) is used for altering the hybridization potential of oligomers and templates in the reaction. Alternatives or additional components include DMSO (dimethyl sulfoxide), proline, trehalose and ionic liquids including imidazolium, pyridinium, pyrrolidinium, and phosphonium, with the anions including halides, tetrafluoroborate
   (BF₄⁻), hexafluorophosphate (PF₆⁻), and bis[(trifluoromethyl) sulfonyl] imide.

In Step 20, 40 μL of plant lysate from above is added to the lyophilized pellet (total volume is about 40 μL) using an exact volume pipette to rehydrate the pellet. An exact volume capillary may also be used for sample transfer. If the reactant mixture from Step 18 was not lyophilized, the liquid and the lysate are mixed.

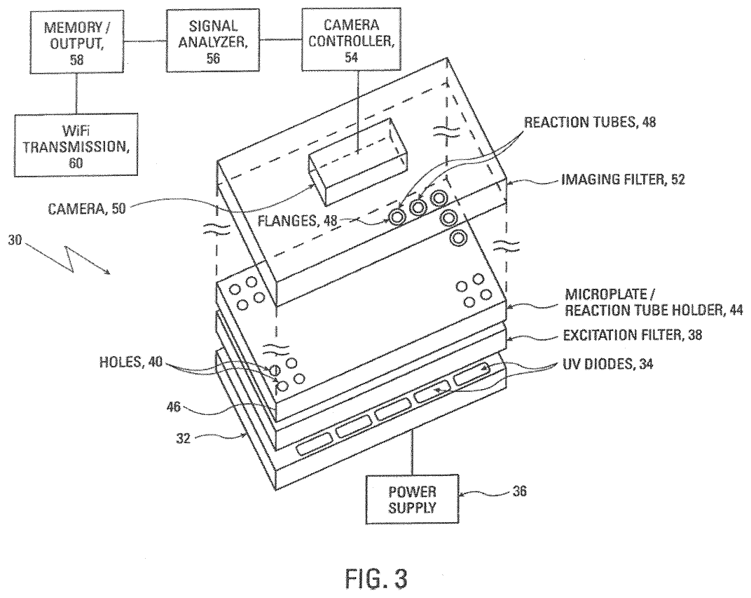

In Step 22, the lysate and reactants are incubated using a dry heat block for about 90 min. (between about 30 min. and about 90 min. has been found to be adequate) at about 65° C. (any temperature between about 60° C. and about 70° C.), after which the reaction is cooled to room temperature (about 21° C.) for about 5 min., as seen in Step 24, such that unincorporated fluorophore-conjugated oligonucleotides are re-annealed to complementary quencher oligonucleotides and can no longer produce visible fluorescence. It should be mentioned that any heating device capable of maintaining the reaction at the chosen constant temperature, such as a water bath, PCR apparatus, as examples, can be used. Fluorophore-conjugated oligonucleotides that have already been incorporated into an amplicon will not be available to re-anneal to complementary quencher oligonucleotides, and will produce visible fluorescence under ultraviolet light. In Step 26, reactions are placed on their side, and viewed with a commercially available (Benchmark Scientific Accuris Instruments) ultraviolet transilluminator viewing device having a broad range of ultraviolet light centered at about 302 nm. A visualization box, having a small viewing hole at the top and fitted with a safe-viewing uv blocking lens (for eye protection) provides a dark environment, and may be used for viewing the fluorescence emissions by eye, or for image photographing using a mobile telephone or camera, or the fluorescence may be captured and analyzed by an embodiment of the electronic image acquisition system of the present invention for viewing fluorescence results from one or more reactions, as illustrated in FIG. 3 and described in EXAMPLE 3. Thus, the test results can be observed visually, or photographed and analyzed using software generated for this purpose. The fluorescence is interpreted as positive for a viral pathogen or other target signal if the reaction glows in a spectrum from red to orange to yellow, as positive for the HLVd signal and negative for viral pathogen signals if the reaction glows green, and with no colored fluorescence in a reaction interpreted as a failed reaction. The fluorophores and the ratios of primer sets were chosen such that the green (Cannabis) fluorescence does not overpower the red (HLVd). Therefore, if there are detectable levels of viral target, the combination of both fluorescence will either show fully red (for viral), or a red spectrum (for the combination), but not fully green. In use, about 1.5 times as much of the virus primer set as the Cannabis primer set was found to achieve this effect, since the HLVd sequence amplifies efficiently and not as much primer is required to overpower the Cannabis target. Examples of observable fluorescence (or lack thereof) may be made available to a user by supplying (a) Positive control reactions containing both Cannabis and HLVd; (b) Negative control reactions only containing Cannabis RNA; and (c) Reactions containing no RNA, with each set of reaction tubes in a kit, as will be described in more detail below.

Step 28, fluorescence may be documented by a digital photograph, a camera, light box, or electronic image acquisition system.

Having generally described embodiments of the invention, the following EXAMPLES demonstrates further aspects thereof.

### Example 1

### A. Selection of Primers for Steps 14a, 14b, and 16:

Primers Specific to HLVd and Cannabis Control Targets are set forth in TABLE 1, and were designed using the Primer Explorer V4 software specifically for designing optimized LAMP primers, and synthesized by a contractor, for amplifying the selected target regions used to detect the HLVd viroid sequence having GenBank RefSeq GCF 000856285.1 and the Cannabis standard reference control sequence NCBI XM 030654946.1. The primer sequences were predicted to specifically amplify target regions without off-target interactions, and/or lack of negative interactions with either other included primers or other biological sample genetic material.

### B. Selection of Fluorophores for Steps 14a and 14b:

To allow efficient amplification of target sequences, while enabling visualization, oligomeric nucleotide conjugated fluorophores (bound to the Forward Internal Primer, FIP, as set forth above) were selected to allow visualization by eye using a single ultraviolet light source for generating fluorescence. This is accomplished by utilizing two individual fluorophores that can each be excited with a single UV light source, but that emit two distinct wavelengths, which can be simultaneously seen by eye. Fluorophores are also selected such that they do not inhibit or hinder sequence amplification from the specific targets of interest. Fluorophore combinations may consist of any commercially available visible conjugated (chemically bonded, conjugated, or attached to the oligonucleotide during synthesis) fluorophore. The number of selected fluorophores can range from one to many (>5). A single fluorophore or multiple fluorophores can be used to target multiple pathogens or multiple sequences in a single pathogen, from the same assay. The assay may or may not contain a separate fluorophore that targets a control host gene/transcript. Wavelength filters may be employed, whereby the emission from one fluorophore is observed at a time. Any host gene DNA or RNA may be used as a target for the internal control. The Cannabis gene was selected because it is well-expressed and is a commonly used.

### C. Selection of Quenchers for Steps 14a, 14b, and 16:

To quench fluorescence from unincorporated primers, quencher oligomers are selected to contain the appropriate conjugated molecule effective for quenching the specific wavelength of fluorescence emitted from the fluorophores used in the assay. Quencher oligonucleotide sequences are chosen to have a specific length, and may contain mismatches, which allow binding of the quencher oligomer to a particular fluorescently labelled oligomer at low temperatures (<50° C.), but not at high temperature (>50° C.).

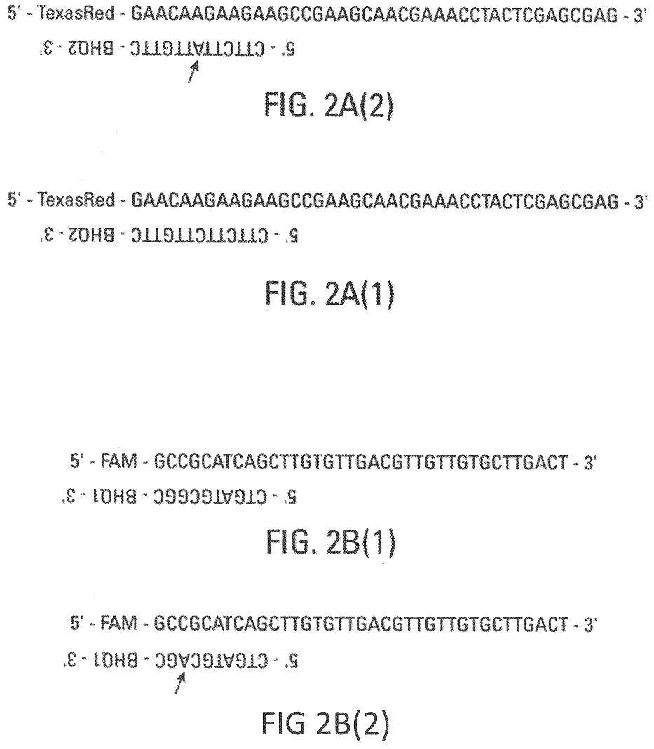

FIG. 2A(1) illustrates the hybridization of the reverse complimentary sequence for the HLVd Forward Internal Primer, FIP, conjugated at the 3′ end to a dark quencher, BHQ2, for the TexasRed fluorophore (586 nm excitation peak utilized) of SEQ ID NO: 8, with the FIP primer conjugated to the TexasRed fluorophore of SEQ ID NO: 2, and FIG. 2A(2) illustrates the hybridization of the reverse complimentary sequence for the HLVd Forward Internal Primer, FIP, conjugated at the 3′ end to a dark quencher, BHQ2, for the TexasRed fluorophore of SEQ ID NO: 9, with the FIP primer conjugated to the TexasRed fluorophore of SEQ ID NO: 2, showing the mismatch marked by the arrow, of an A (adenine) nucleotide bound to a G (guanine) nucleotide instead of a C (cytosine) nucleotide bound to the G (guanine) nucleotide; while FIG. 2B(1) illustrates the hybridization of the reverse complimentary sequence for the Cannabis Forward Internal Primer, FIP, conjugated at the 3′ end to a dark quencher, BHQ1, for the FAM fluorophore (493 nm excitation peak) of SEQ ID NO: 16, with the FIP primer having SEQ ID NO: 10 conjugated to the FAM fluorophore, and FIG. 2B(2) illustrates the hybridization of the reverse complimentary sequence for the Cannabis Forward Internal Primer, FIP, conjugated at the 3′ end to a dark quencher, BHQ1, for the FAM fluorophore of SEQ ID NO: 17, with the FIP primer having SEQ ID NO: 10 conjugated to the FAM fluorophore, showing the mismatch marked by the arrow, of an A (adenine) nucleotide bound to a C (cytosine) nucleotide instead of a G (guanine) nucleotide bound to the C (cytosine) nucleotide. The identified mismatches weaken the hybridization/binding of the primers bearing the fluorophores with the primers bearing the dark quenchers.

When reverse complementary sequences hybridize, “mismatches” mean any non-Watson-Crick base-pairing pairs. Mismatches alter the hybridization strength of the duplex, making it weaker, and, along with the chosen length of the quencher, produce the effect of temperature on binding. That is, binding of the quencher to its target fluorophore becomes possible at low temperatures, while the two oligonucleotides separate at higher temperatures, such that at the assay temperature, RT-LAMP amplification can occur. Online programs such as those available on the websites of Sigma or IDT were used to predict suitable quenchers based on sequence and calculated hybridization strength, delta G, but their actual operation must be tested empirically. Therefore, quencher oligonucleotide sequences are selected such that they do not inhibit amplification of either the target pathogen or the host control transcript/gene. Quencher conjugated oligonucleotides were purchased from commercial sources, with and without mismatches, and with a variety of lengths, in order to optimize their properties. As stated above, the alternate (mismatched) quenchers generated essentially the same test results.

Other quenchers that may be used include: (a) IowaBlack-FQ, which can quench the FAM fluorophore in the Cannabis target; (b) IowaBlack-RQ, which can quench the TexasRed fluorophore in the HLVd target; (c) TAMRA, which can quench the FAM fluorophore; and (d) BlackBerry Quencher 650, which can quench the TexasRed fluorophore, as examples. The FAM and TexasRed fluorophores, and the above quenchers conjugated to oligonucleotides are commercially available from multiple companies including: IDT, GeneWiz, ThermoFisher, Abcam, and Biosynthesis, as examples.

### D. Optimization of Primers for Steps 14a, 14b, and 16:

The ratios of different primer sets targeting multiple sequences for allowing amplification of all targets, and for facilitating visualization of all amplified targets, such that complete quenching of fluorescence from unincorporated fluorescently conjugated oligomers occurred, was determined experimentally. RTLAMP primer sets consist of 6 oligomers for targeting each region of interest. Primer and quencher aqueous solutions were mixed together at an experimentally determined ratio of 1:2 of primer to quencher. Other ratios that may yield usable results can range from 1:1 to 1:2.5 of primer to quencher. This mixture was heated to about 85° C. for about 2 min. to remove any secondary structure of the oligonucleotides, and slowly cooled to room temperature. Slow cooling allows the quencher to bind or anneal to the primer. Step 14b illustrates the pre-annealing step for the Cannabis primer SEQ ID NO: 10 (FIP-FAM) and quencher primer SEQ ID NO: 16 (Quencher1) in a different tube in a 1:1 ratio, and was identically and separately performed from the pre-annealing of the FIP/Quencher combination for the HLVd FIP and quencher set forth above in Step 14a, and forming Mixture 1. A second mixture, Mixture 2, was pre-annealed in another tube using SEQ ID NO: 10 (FIP-FAM) and SEQ ID NO: 17 (Quencher2) in a ratio 1:1.5 of FIP-FAM to Quencher2. Both Mixture 1 and Mixture 2 were separately heated to about 85° C. for about 2 min. to remove any secondary structure of the oligonucleotides, and slowly cooled to room temperature. Both Mixture 1 and Mixture 2 for the Cannabis were used in the final reaction at a ratio of 1.3 (Mixture 1:Mixture 2).

The ratio that gave efficient amplification from all targets and allowed visual analysis of the results was found to be 1.5 of HLVd primers to Cannabis primers. However, ratios of HLVd to Cannabis primers between 0.5 and 2.5 may also produce a usable result. Considerations for this process included: transcription level of pathogen(s) target verses control target, expected pathogen load in the sample compared to the target, brightness of selected fluorophores, ease of visualization of different wavelengths by the human eye (i.e., green is more readily seen than red) or analyzed using software, and the number of different sequences targeted by the same fluorophore in the assay.

In an RT-LAMP reaction the first step is conversion of the RNA to complementary DNA (cDNA) carried out by an RT enzyme included in the reaction. The oligomers that prime this process are the same reverse primers that catalyze the loop mediated amplification by the polymerase in the next step. However, the use of quencher technology also means that a reverse complement sequence that can bind the target RNA template itself is introduced. Binding of free quencher to the RNA template prior to heating the reaction can lead to reaction inhibition because the interaction blocks reverse transcription of the sequence needed to initiate amplification. Additionally, free primers can initiate non-specific amplification at low temperatures in all LAMP reactions. Therefore, the reaction components need to be mixed immediately prior to running the reaction, which is what is customarily done. Prehybridization (pre-annealing) steps (heating together and slow-cooling to promote annealing) the fluorophore-conjugated oligomers (SEQ ID NO: 2 and SEQ ID NO: 10) with quencher oligomers (SEQ ID NO: 8 or SEQ ID NO: 9, and Seq ID NO: 16 or SEQ ID NO: 17, respectively) were optimized to yield efficient quenching in the absence of targets. That is, temperature and timing for prehybridization were optimized to maintain quenching without inhibiting amplification of the chosen targets (HLVd and Cannabis) in the assay, thereby reducing false positives and false negatives. By adding the pre-annealing step, the free quencher and primers are “locked” together and resulting duplex becomes “hot-start”. Without this added process the reaction does not amplify. This significantly reduces non-specific amplification allowing the present chemistry to be stable for days at room temperature or longer, if chilled, as a pre-mixed, liquid reaction solution, and up to a year as a lyophilized pellet.

Summarizing, without the pre-annealing step several negative consequences occur:
a. The FIP labelled primer is not fully quenched at the start of the reaction, thereby increasing background florescence;
b. Labelled FIP primers are not “blocked” at room temperature from binding the template (since they might not be fully bound by quencher) increasing the chances of non-specific amplification;
c. Excess free (unbound) FIP or quencher (for some quencher sequences) may bind target nucleic acid sequences and preclude binding of the labeled FIP (or other primers), thereby inhibiting the reaction; and
d. Without the pre-annealing step, the reaction does not proceed, likely due to point c. above.
   Although the pre-annealing step accomplishes many things, it may not be strictly necessary for every target.

Further, if the reaction continues to react after the designated time, the visual readout can change from negative to positive, since the enzymes are still available and there is an increased opportunity that they can locate an off-target species and beginning amplification of that species. Thus, if left to react for long periods all such analyses will likely become positive. To reduce this possibility, LAMP enzymes are often inactivated at the end of the designated reaction time by heating the reaction to a high temperature (around 85° C.) for 5-15 minutes, which destroys the enzymes and stops such reactions from progressing. This additional step is inconvenient for the present on-site assay, where a thermal cycler is generally not available. Effectively quenching the fluorophore FIP primer once the reaction time is over by slow-cooling to room temp accomplishes this result, and if the enzymes still remain active, a false positive is not observed, since the read-out is fluorescence and fluorescence from unincorporated labeled oligonucleotides is quenched. Further, because the detection technology is tied to incorporation of a fluorescent primer (versus the more standard methods such as intercalating dyes or pH indicators), non-specific amplification involving most primer-dimer combinations will not trigger a positive result even if spurious amplification occurs in the background.

### E. Optimization of Reaction Conditions for Steps 20 and 22:

In Step 22, the lysate and reactants are incubated using a dry heat block for about 90 min. (between about 30 min. and about 90 min. has been found to be adequate) at about 65° C. (any temperature between about 60° C. and about 70° C.), after which the reaction is cooled to room temperature (about 21° C.) for about 5 min., as seen in Step 24, such that unincorporated fluorophore-conjugated oligonucleotides are re-annealed to complementary quencher oligonucleotides and can no longer produce visible fluorescence.

### Example 2

TABLE 5 shows the results of a blind study to determine the accuracy of the present method compared to conventional RT-qPCR for the detection of hop latent viroid (HLVd) in Cannabis plant tissue. The results were evaluated by eye and recorded by the operator. Colors between yellow (Positive-Low HLVd Level) and red (Positive-High HLVd Level) were recorded as positive, bright green as negative, and dark/non-fluorescent samples were recorded as failed. Note that the positive-medium HLVd Level appeared as an orange color. TABLE 5 shows that at the concentrations of plant tissue and pathogen tested, the present method performed with 100% accuracy as compared to conventional qRT-PCR, when evaluated by eye.

TABLE 5

| | | | | | | | Estimated |
|---|---|---|---|---|---|---|---|
| | | | Average | | HLVd | | Viroid |
| | | | Control | | Target | | Load |
| Sample | Samples | Percentage | CT | St. | CT | St. | (genomes |
| Type | Tested | Positive | (PCR) | Dev. | (PCR) | Dev. | per μL)* |
| Positive - | 15 | 100% | 28.5 | 1.2 | 24.0 | 0.5 | 2649.2 |
| High | | | | | | | |
| HLVd | | | | | | | |
| Level | | | | | | | |
| Positive - | 15 | 100% | 30.7 | 0.9 | 27.4 | 1.3 | 502.4 |
| Medium | | | | | | | |
| HLVd | | | | | | | |
| Level | | | | | | | |
| Positive - | 15 | 100% | 31.2 | 0.2 | 31.4 | 1.9 | 27.8 |
| Low | | | | | | | |
| HLVd | | | | | | | |
| Level | | | | | | | |
| Negative - | 20 | 0% | 32.67 | 0.8 | NaN | NA | NA |
| No | | | | | | | |
| HLVd | | | | | | | |
| NTC - | 30 | 0% | NaN | NA | NaN | NA | NA |
| Failed (no | | | | | | | |
| tissue | | | | | | | |
| added) | | | | | | | |

*Viroid load was determined using standard curve data collected from a serial dilution performed in triplicate.
CT indicates cycle threshold.

### Example 3

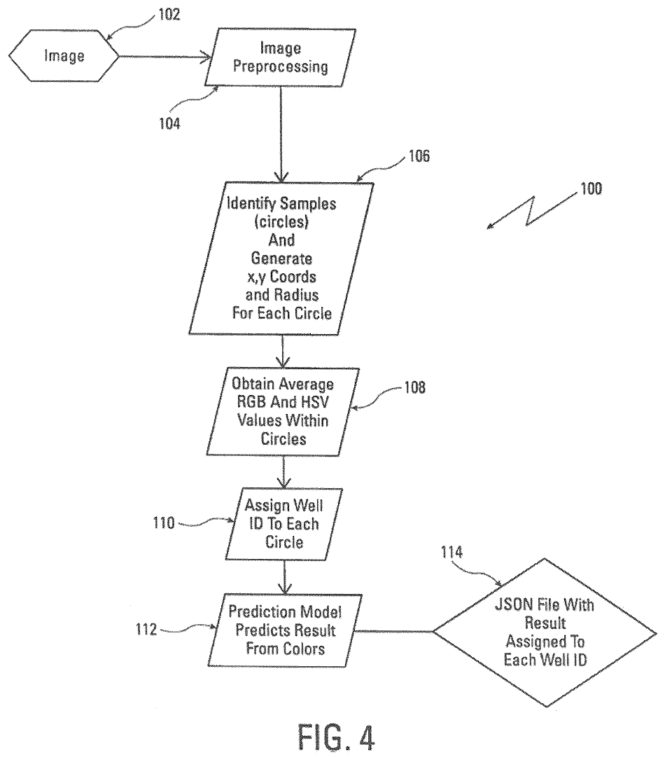

FIG. 3 is a schematic representation of electronic image acquisition system, 30, for viewing fluorescence results from one or more reactions. Ultraviolet source, 32, shown as having at least one ultraviolet diode (UV LED), 34, powered by power supply, 36, directs UV radiation through excitation filter, 38, and into reaction tubes, 40. Reaction tubes 40, are standard PCR tubes fabricated from polypropylene, having lids, and adapted to fit through holes, 42, in microplate/reaction tube holder, 44. Microplate 44 has a thickness, 46, such that a portion of each of tubes 40 is exposed to UV radiation. In experiments, approximately the bottom half of each of the reaction tubes is directly exposed. This permits UV radiation from UV source 32 to generate fluorescence in the fluorophores contained therein. Reaction tubes 40 may be held in place in holes 42 by integral external flanges, 48, formed thereon, and/or by tapering of the holes 42 or the reaction tubes 40 themselves, or a combination of tapered surfaces. An image of fluorescence emanating from the top portion of reaction tubes 40 is taken by camera, 50, after the fluorescence passes through imaging filter, 52. Camera 50 is operated by camera controller, 54, the output of which may be directed into signal analyzer, 56, and the acquired image may be stored and/or visualized using memory/output device, 58. Images may be transmitted to users using Wi-Fi, 60. Currently, the data is analyzed outside image acquisition system 30, after storage 58 and Wi-Fi transmission 60. However, it is anticipated that the analysis algorithm set forth in FIG. 4 may be implemented in system 30, as shown in FIG. 3. UV LEDs 34 are used to excite both FAM and Texas Red fluorophores in reaction tubes 40. LEDs having a UV radiation peak at 308±5 nm were found to be most effective at generating bright, saturated red and green fluorescence, rendering the images more readily interpreted. Excitation filter 38 transmits UV light for the excitation of fluorophores and blocks visible light (420 nm-650 nm), thereby improving image contrast, while imaging filter 52 blocks visible light emitted by UV LEDs 34 at shorter wavelengths of the visible light spectrum (<430 nm).

FIG. 4 shows algorithm, 100, for analyzing images taken by camera 48. In Step, 102, camera 48 captures an image of reaction tube holder 44 showing fluorescence from reaction tubes 40. Step 104 preprocesses the image, which may include the steps of masking colors outside of expected RGB range (colors constructed from the combination of the Red, Green, and Blue colors), and applying a blur to image to reduce noise. The need for masking arises from the occasional observation of particulates (hair, lint, etc.) in the image, that can contribute to the fluorescence. If these particulates are not removed from the image, their color values (RGB and HSV (color model used for image analysis, where H stands for Hue, the color portion of the model; S represents Saturation, the amount of gray in a particular color; and V describes the brightness or intensity of the color)) will be included in the algorithm. The color values of the particulates have been coded so any pixel that contains these color values will be masked and not included in the algorithm.

A Hough circle transform is used to identify samples in the fluorescence image, allowing circular objects to be extracted and the location (X, Y) of each circular object to be identified. In the present situation, each circle identified must correspond to a sample. However, when raw, unprocessed images were used, the Hough transformer located multiple circles within each sample; that is, circles were drawn around condensation and spots of fluorescence, anything that had an edge in the image. To cure this problem, edges were reduced by applying a median blur, which takes all of the pixel values within a defined area and replaces them with the median in the defined area. This “smooths” the fluorescence image and permits the circle transform to accurately identify all the samples in the image,

The processed image is assigned circles, each having x and y coordinates at its center, and a radius in Step 106, and samples are identified using circles that fit a defined size range, one circle corresponding to one sample in the reaction plate. Average HSV and RGB values are obtained from the circles in Step 108, and a well identification is assigned to each circle in Step 110. The Prediction model, Step 112, is a multinomial logistic regression model using a “one vs rest” strategy to predict the results from these colors. The “one vs rest” strategy requires a model to be created for each class (Negative, Positive, and Failed). The 3 models were “trained” using RGB and HSV values from 300 positive samples, 300 negative samples, and 200 failed samples. When a new sample is input into the models, the color values (RGB, HSV) of that sample are used to determine which class (result) the data most closely resembles. Each of the 3 models predicts a class membership probability for that sample and the class with the highest probability is assigned to the sample.

The results are stored in the database as a JSON file (JavaScript Object Notation) in Step 114, and consist of the well ID and the highest probability that corresponds to that well ID.

### Example 4

Embodiments of the present method for detecting hop latent viroid nucleic acid may be included in a kit for reverse transcription loop-mediated isothermal amplification and fluorescent detection of the pathogen nucleic acid, along with a Cannabis control gene, from biological samples, including plant tissue extract, using the specific oligonucleotide primers, fluorophore-labeled probes, buffers, enzymes, and quenchers set forth above. Pre-barcoded, screw-capped collection tubes containing sample purification components in an optimization buffer and instructions are packaged together for sample collection intended to be performed at the cultivation facility/site.

A kit may include: (A) at least one 5 mL volume screw-capped tube having solid filtration components (for example, prewashed activated charcoal and Chelex 100 (50-100 mesh) Resin) in a sample optimization buffer and a sticker label with an identifier code. A described amount of plant tissue comprising of root, leaf or stem is added directly to sample collection tube, the lid is closed tightly and the tube is agitated for several seconds to mix the components (B) At least one fixed-volume micropipette, or a fixed-volume capillary tube having a plunger (for example, a capillary action transfer stick) or bulb, capable of transferring a 40 μL sample volume and associated pipette tips; (C) a 0.2 mL strip or a microplate, or at least one optically-clear 0.1 mL volume reaction tube that may be attached in groups of 8 tubes, each tube having individual attached snap cap, being capable of withstanding the elevated temperature of about 65° C. and remain sealed, and containing a lyophilized reaction sphere or pellet (Note that the chemistry could work using a frozen liquid, but then, a much smaller amount of sample than 40 μL would have to be used, thereby decreasing the sensitivity of the test.); (D) a dry heating block with or without a heated lid or water bath capable of being heated for heating the reaction tubes to about 65° C.; and (E) an apparatus for visualization of the reactions. This latter apparatus may include a source of UV radiation, a visualization box, having a small viewing hole at the top and fitted with a safe-viewing UV blocking lens for providing a dark environment, for viewing the fluorescence emissions by eye, or for image photographing using a mobile telephone or camera, or the fluorescence may be captured and analyzed by an embodiment of the electronic image acquisition system of the present invention for viewing fluorescence results from one or more reactions, as illustrated in FIG. 3 and described in EXAMPLE 3.

The sample optimization buffer may include Tris-HCl buffer (pH 8), EDTA, Trehalose, BSA (Bovine Serum Albumin), and Tween-20. The reaction tubes may include: DNA polymerase, reverse transcriptase, deoxyadenosine triphosphate, deoxycytidine triphosphate, deoxyguanosine triphosphate, deoxythymidine triphosphate, BIP, F3, B3, Loop F, and Loop B first oligonucleotide primers for hybridizing with the HLVd nucleic acid sequence, BIP, F3, B3, Loop F, and Loop B second oligonucleotide primers for hybridizing with the Cannabis gene nucleic acid control, an annealed first FIP primer conjugated to a first fluorophore and having SEQ ID NO: 1, with a first reverse complementary oligonucleotide conjugated to a first quencher for the first fluorophore and having SEQ ID NO: 13, an annealed second FIP primer conjugated with a second fluorophore and having SEQ ID NO: 7, with a second reverse complementary oligonucleotide conjugated to a second quencher for the second fluorophore and having SEQ ID NO: 14, magnesium sulfate, Tris(hydroxymethyl)aminomethane hydrochloride, ammonium sulfate, potassium chloride, and Tween-20. Effective concentrations of these compositions for reaction with 40 μL of sample volume are lyophilized (freeze dried) into pellet or spherical form and placed into the reaction tubes for use in the kits.

Kits may include: (a) An apparatus kit containing: a sample collection tube holder, a heating block, at least one black reaction plate for holding reaction tubes for fluorescence visualization, and a device for exciting and viewing fluorescence; (b) a testing kit containing: at least one tube having a cap and solid filtration components in a sample optimization buffer, at least one fixed-volume micropipette; or a fixed-volume capillary tube having a plunger, and at least one optically-clear reaction tube, each tube having individual attached snap cap, being capable of withstanding the elevated temperature of about 65° C. and remain sealed, and containing a lyophilized reaction sphere or pellet; and (c) an apparatus kit containing: a sample collection tube holder; a heating block, at least one black reaction plate for holding reaction tubes for fluorescence visualization, and a device for exciting and viewing fluorescence; and a testing kit containing: at least one screw-capped tube having solid filtration components in a sample optimization buffer, at least one fixed-volume micropipette, or a fixed-volume capillary tube having a plunger, and at least one optically-clear reaction tube, each tube having an individual cap, being capable of withstanding the elevated temperature of about 65° C. and remain sealed, and containing a lyophilized reaction sphere or pellet.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the appended claims.

What is claimed is:

1. A method using RT-LAMP amplification for detecting RNA from hop latent viroid infecting a Cannabis plant, comprising:
collecting a sample of tissue from the Cannabis plant to be analyzed and containing a plant tissue control;
mixing the Cannabis plant tissue with a lysing agent, a DNA/RNA stabilizer, a component for minimizing amplification inhibitors, and a pH buffer, thereby forming a plant lysate;
selecting a first oligonucleotide primer from the primers FIP, BIP, F3, B3, Loop F, and Loop B for hybridizing to the hop latent viroid nucleic acid sequence, the selected first oligonucleotide primer being conjugated to a first fluorophore at its 5′ end, and a first fluorescence quencher for the first fluorophore conjugated to the 3′ end of a first reverse complementary oligonucleotide sequence to the selected first oligonucleotide primer;
selecting a second oligonucleotide primer from the primers FIP, BIP, F3, B3, Loop F, and Loop B for hybridizing to the plant lysate nucleic acid sequence, the selected second oligonucleotide primer being conjugated to a second fluorophore at its 5′ end, and a second fluorescence quencher for the second fluorophore conjugated to the 3′ end of a second reverse complementary oligonucleotide sequence to the selected second oligonucleotide primer;
pre-annealing the first oligonucleotide primer to the first reverse complementary oligonucleotide sequence thereof, and the second oligonucleotide primer to the second reverse complementary oligonucleotide sequence thereof;
forming a RT-LAMP amplification solution, comprising:
the plant lysate;
the primers FIP, BIP, F3, B3, Loop F, and Loop B for hybridizing to the hop latent viroid nucleic acid sequence that were not selected;
the primers FIP, BIP, F3, B3, Loop F, and Loop B for hybridizing to the nucleic acid sequence for the plant transcript control that were not selected;
the pre-annealed first primer to the first reverse complementary oligonucleotide for the first primer for said hop latent viroid; and
the pre-annealed second primer to the second reverse complementary oligonucleotide for the second primer for said plant lysate;
heating the amplification solution for a chosen time at a single chosen temperature, such that said RT-LAMP amplification reaction takes place for both the hop latent viroid and the plant lysate;
cooling the amplification solution following said RT-LAMP amplification reaction;
illuminating the cooled amplification solution with ultraviolet light, having chosen wavelengths such that the first fluorophores and the second fluorophore incorporated into products formed in said RT-LAMP amplification reaction emit fluorescence radiation having specific wavelengths; and
observing and interpreting the specific wavelengths of the fluorescence radiation.

2. The method of claim 1, wherein the ratio of the first oligonucleotide primer to the first reverse complementary oligonucleotide sequence thereof is 1:2, and the ratio of the second oligonucleotide primer to the second and a third reverse complementary oligonucleotide sequence thereof, is 1:1 and 1.5, respectively.

3. The method of claim 2, wherein the RT-LAMP amplification solution includes: a strand displacement DNA polymerase, a reverse transcriptase, deoxyadenosine triphosphate, deoxycytidine triphosphate, deoxyguanosine triphosphate, deoxythymidine triphosphate, magnesium sulfate, trimethylglycine, ammonium sulfate, Tris(hydroxymethyl)aminomethane hydrochloride, a polysorbate nonionic surfactant, Betaine, dUTP and thermolabile uracil DNA glycosylase, the annealed first primer with the first reverse complementary oligonucleotide thereof, and the annealed second primer with the second reverse complementary oligonucleotide thereof.

4. The method of claim 1, wherein the first primer has the sequence of SEQ ID NO: 1, and the first reverse complimentary oligonucleotide has the sequence of SEQ ID NO: 8 or SEQ ID NO: 9.

5. The method of claim 1, wherein the first fluorophore comprises: Texas Red, and the first fluorescence quencher is chosen from BHQ2, IowaBlack-RQ, BlackBerry Quencher 650, and other quencher molecules effective for quenching light in the red spectrum.

6. The method of claim 1, wherein the second oligonucleotide primer has the sequence of SEQ ID NO: 10, and the second reverse complimentary oligonucleotide has the sequence of SEQ ID NO: 16 or SEQ ID NO: 17.

7. The method of claim 1, wherein the second fluorophore comprises FAM, and the second fluorescence quencher is chosen from BHQ1, IowaBlack-FQ, TAMRA, and other quencher molecules effective for quenching light in the green spectrum.

8. The method of claim 1, wherein the RT-LAMP amplification solution further comprises deoxyuridine triphosphate.

9. The method of claim 1, wherein the RT-LAMP amplification solution further comprises Antarctic thermolabile uracil DNA glycosylase.

10. The method of claim 1, wherein said steps of illuminating the cooled amplification solution with ultraviolet light having chosen wavelengths, and observing and interpreting the specific wavelengths of the fluorescence radiation are performed using an image acquisition system.

11. The method of claim 1, wherein the plant lysate includes prewashed, activated charcoal and Chelex Resin.

12. The method of claim 1, wherein the plant lysate includes Tris-HCl buffer, EDTA, Trehalose, BSA, and Tween-20.

13. The method of claim 1, wherein said step of heating the amplification solution is performed for about 90 min., and the single chosen temperature is about 65° C.

14. The method of claim 1, wherein said step of cooling the amplification solution following said RT-LAMP amplification reaction is performed for about 5 min. at a temperature of about 21° C.

15. The method of claim 1, wherein the ratio of first oligonucleotide primers to second oligonucleotide primers in the RT-LAMP amplification solution is 1.5.

16. The method of claim 1, wherein said step of observing the specific wavelengths of the fluorescence radiation is performed by visual inspection, a red, yellow, or orange color indicating the presence of hop latent viroid, a greenish color indicating the absence of the hop latent viroid, and the absence of fluorescence indicating a failed amplification.

17. The method of claim 10, wherein performing said step of observing the specific wavelengths of the fluorescence radiation is achieved using an image acquisition system for at least one reaction tube responsive to at least one fluorophore therein exposed to UV radiation, comprising:
a reaction tube holder, having a top surface and a bottom surface parallel thereto, a selected distance therebetween, and a chosen number of holes formed between the top surface and the bottom surface, whereby a portion of the at least one reaction tube having a chosen length and placed in a selected hole extends below the bottom surface when the top of the at least one reaction tube is positioned in the vicinity of the top surface;
a source of ultraviolet radiation for irradiating the portion of the at least one reaction tube extending below the bottom surface;
an excitation filter for selecting a wavelength range of ultraviolet radiation from said source of ultraviolet radiation effective for exciting fluorescence excitation in the at least one fluorophore of the at least one reaction tube;
an imaging filter for blocking ultraviolet radiation and transmitting fluorescence excitation from the at least one fluorophore;
a camera for receiving the transmitted fluorescence excitation from the at least one fluorophore; and
a camera controller for identifying the location of the selected hole for the at least one reaction tube.

18. The method of claim 17, wherein said source of ultraviolet radiation comprises at least one light emitting diode having an ultraviolet radiation peak at 308±5 nm.

19. The method of claim 17, wherein said excitation filter transmits ultraviolet radiation, and blocks visible light between 420 nm and 650 nm.

20. The method of claim 17, wherein said imaging filter blocks visible light emitted by the at least one light emitting diode at wavelengths <430 nm.

21. A kit for detecting hop latent viroid, HLVd, nucleic acid infecting a Cannabis plant by RT-LAMP amplification, comprising:
at least one sample collection tube having a first chosen volume and a cap, and containing a lysing agent, a DNA/RNA stabilizer, a component for minimizing amplification inhibitors, and a pH buffer;
at least one fixed-volume micropipette, or at least one fixed-volume capillary tube having a plunger or bulb;
at least one optically clear reaction tube having a second chosen volume, each tube having a cap, and containing a RT-LAMP amplification solution;
a dry heating block or a water bath capable of being heated, for heating the at least one optically clear reaction tube to about 65° C.;
at least one reaction plate capable of holding at least one reaction tube; and
apparatus for irradiating the at least one reaction tube with ultraviolet light, and for visualization of resulting fluorescence.

22. The kit of claim 21, wherein said apparatus for irradiating the at least one reaction tube with ultraviolet light, and for visualization of resulting fluorescence, comprises: a visualization enclosure having a small hole fitted with an ultraviolet filter for permitting visualization of fluorescence from the at least one reaction tube by eye or by photography using a mobile telephone or camera.

23. The kit of claim 21, wherein said apparatus for irradiating the at least one reaction tube with ultraviolet light, and for visualization of resulting fluorescence, comprises an image acquisition system, comprising:
a reaction tube holder, having a top surface and a bottom surface parallel thereto, a selected distance therebetween, and a chosen number of holes formed between the top surface and the bottom surface, whereby a portion of the at least one reaction tube having a chosen length and placed in a selected hole extends below the bottom surface when the top of the at least one reaction tube is positioned in the vicinity of the top surface;
a source of ultraviolet radiation for irradiating the portion of the at least one reaction tube extending below the bottom surface;
an excitation filter for selecting a wavelength range of ultraviolet radiation from said source of ultraviolet radiation effective for exciting fluorescence excitation in the at least one fluorophore of the at least one reaction tube;
an imaging filter for blocking ultraviolet radiation and transmitting fluorescence excitation from the at least one fluorophore;
a camera for receiving the transmitted fluorescence excitation from the at least one fluorophore; and
a camera controller for identifying the location of the selected hole for the at least one reaction tube.

24. The kit of claim 21, wherein said RT-LAMP amplification solution includes: a strand displacement DNA polymerase, a reverse transcriptase, deoxyadenosine triphosphate, deoxycytidine triphosphate, deoxyguanosine triphosphate, deoxythymidine triphosphate, magnesium sulfate, trimethylglycine, ammonium sulfate, Tris(hydroxymethyl)aminomethane hydrochloride, a polysorbate nonionic surfactant, Betaine, dUTP and thermolabile uracil DNA glycosylase, the annealed first primer with the first reverse complementary oligonucleotide thereof, and the annealed second primer with the second reverse complementary oligonucleotide thereof.

25. A kit for detecting hop latent viroid, HLVd, nucleic acid infecting a Cannabis plant by RT-LAMP amplification, comprising:
at least one sample collection tube having a first chosen volume and a cap, and containing a lysing agent, a DNA/RNA stabilizer, a component for minimizing amplification inhibitors, and a pH buffer;
at least one fixed-volume micropipette, or at least one fixed-volume capillary tube having a plunger or bulb; and
at least one optically clear reaction tube having a second chosen volume, each tube having a cap, and containing a RT-LAMP amplification solution.

26. The kit of claim 25, wherein said RT-LAMP amplification solution includes: a strand displacement DNA polymerase, a reverse transcriptase, deoxyadenosine triphosphate, deoxycytidine triphosphate, deoxyguanosine triphosphate, deoxythymidine triphosphate, magnesium sulfate, trimethylglycine, ammonium sulfate, Tris(hydroxymethyl)aminomethane hydrochloride, a polysorbate nonionic surfactant, Betaine, dUTP and thermolabile uracil DNA glycosylase, the annealed first primer with the first reverse complementary oligonucleotide thereof, and the annealed second primer with the second reverse complementary oligonucleotide thereof.

27. A method using RT-LAMP amplification for detecting RNA from hop latent viroid infecting a Cannabis plant, comprising the steps of:
collecting a sample of the Cannabis plant tissue containing a plant transcript control;
mixing the plant tissue with solid filtration components for removing amplification inhibitors, and a sample optimization buffer, thereby forming a crude plant lysate;
pre-annealing a first oligonucleotide primer selected from the primers FIP, BIP, F3, B3, Loop F, and Loop B for hybridizing to the hop latent viroid nucleic acid sequence, the selected first oligonucleotide primer being conjugated to a first fluorophore at its 5′ end, with a first fluorescence quencher for the first fluorophore conjugated to the 3′ end of a first reverse complementary oligonucleotide sequence to the selected first oligonucleotide primer, forming an annealed first primer with a first reverse complementary oligonucleotide;
pre-annealing a second oligonucleotide primer selected from the primers FIP, BIP, F3, B3, Loop F, and Loop B for hybridizing to the nucleic acid sequence for the plant transcript control, the selected second oligonucleotide primer being conjugated to a second fluorophore at its 5′ end, with a second fluorescence quencher for the second fluorophore conjugated to the 3′ end of a second reverse complementary oligonucleotide sequence to the selected second oligonucleotide primer, forming an annealed second primer with a second reverse complementary oligonucleotide;
preparing an aqueous solution or lyophilized pellet comprising: a strand displacement DNA polymerase, a reverse transcriptase, deoxyadenosine triphosphate, deoxycytidine triphosphate, deoxyguanosine triphosphate, deoxythymidine triphosphate, dUTP and thermolabile uracil DNA glycosylase, the primers FIP, BIP, F3, B3, Loop F, and Loop B for hybridizing to the hop latent viroid nucleic acid sequence that were not selected in said pre-annealing step for said first primer; the primers FIP, BIP, F3, B3, Loop F, and Loop B for hybridizing to the nucleic acid sequence for the plant transcript control that were not selected in said pre-annealing step for said second primer; the annealed first primer with a first reverse complementary oligonucleotide for the first primer from said pre-annealing step for said hop latent viroid; the annealed second primer with a second reverse complementary oligonucleotide for the second primer from said pre-annealing step for said plant transcript control; magnesium sulfate, trimethylglycine, Tis(hydroxymethyl)aminomethane hydrochloride, ammonium sulfate, and a polysorbate nonionic surfactant;
adding the sample of the Cannabis crude plant lysate containing a plant transcript control to the aqueous solution or lyophilized pellet, forming an amplification solution;
heating the amplification solution for a chosen time at a single chosen temperature, whereby the first primer is separated from the annealed first oligonucleotide primer with a first reverse complementary oligonucleotide from said pre-annealing step for the first primer for hybridizing the hop latent viroid nucleic acid sequence, and whereby the second primer is separated from the annealed second oligonucleotide primer with a second reverse complementary oligonucleotide from said pre-annealing step for the second primer for hybridizing the plant transcript control nucleic acid sequence, such that said RT-LAMP amplification reaction for both the hop latent viroid and the plant transcript control take place;
cooling the amplification solution following said RT-LAMP amplification reactions for a chosen period of time at a chosen temperature, forming a cooled amplification solution, whereby unreacted first reverse complementary oligonucleotides for said hop latent viroid are again annealed to the unreacted first oligonucleotide primers, and whereby unreacted second reverse complementary oligonucleotides for said plant conscript control are again annealed to the unreacted second oligonucleotide primers;
providing an ultraviolet light, wherein the cooled amplification solution is illuminated using the ultraviolet light, and wherein the ultraviolet light has a chosen wavelength such that the first primers and the second primers incorporated into products formed in said RT-LAMP amplification reactions for said hop latent viroid and said plant transcript control emit fluorescence radiation having specific wavelengths; and
observing and interpreting the specific wavelengths of the fluorescence radiation.

28. The method of claim 27, wherein the first primer has a sequence of SEQ ID NO: 1, and the first reverse complimentary oligonucleotide has a sequence of SEQ ID NO: 8 or SEQ ID NO: 9.

29. The method of claim 27, wherein the first fluorophore comprises Texas Red, and the first fluorescence quencher is chosen from BHQ2, IowaBlack-RQ, BlackBerry Quencher 650, and other quencher molecules effective for quenching light in the red spectrum.

30. The method of claim 27, wherein the second oligonucleotide primer has a sequence of SEQ ID NO: 10, and the second reverse complimentary oligonucleotide has a sequence of SEQ ID NO: 16 or SEQ ID NO: 17.

31. The method of claim 27, wherein the second fluorophore comprises FAM, and the second fluorescence quencher is chosen from BHQ1, IowaBlack-FQ, and TAMRA, and other quencher molecules effective for quenching light in the green spectrum.

32. The method of claim 27, wherein the aqueous solution or lyophilized pellet further comprises deoxyuridine triphosphate and thermolabile uracil DNA glycosylase.

33. The method of claim 27, wherein the solid filtration components comprise prewashed, activated charcoal and Chelex Resin, and wherein the sample optimization buffer comprises Tris-HCl buffer, EDTA, Trehalose, BSA, and Tween-20.

34. The method of claim 27, wherein said step of heating the amplification solution is performed for about 90 min., and the single chosen temperature is about 65° C.

35. The method of claim 27, wherein said step of cooling the amplification solution following said RT-LAMP amplification reaction is performed for about 5 min. at a temperature of about 21° C.

36. The method of claim 27, wherein said step of observing the specific wavelengths of the fluorescence radiation is performed by visual inspection, a red, yellow, or orange color indicating the presence of hop latent viroid, and a greenish color indicating the absence of the hop latent viroid.

37. The method of claim 27, wherein said steps of providing an ultraviolet light and observing the specific wavelengths of the fluorescence radiation are performed using an image acquisition system, comprising:
a reaction tube holder, having a top surface and a bottom surface parallel thereto, a selected distance therebetween, and a chosen number of holes formed between the top surface and the bottom surface, whereby a portion of the at least one reaction tube having a chosen length and placed in a selected hole extends below the bottom surface when the top of the at least one reaction tube is positioned in the vicinity of the top surface;
a source of ultraviolet radiation for irradiating the portion of the at least one reaction tube extending below the bottom surface;
an excitation filter for selecting a wavelength range of ultraviolet radiation from said source of ultraviolet radiation effective for exciting fluorescence excitation in the at least one fluorophore of the at least one reaction tube;
an imaging filter for blocking ultraviolet radiation and transmitting fluorescence excitation from the at least one fluorophore;
a camera for receiving the transmitted fluorescence excitation from the at least one fluorophore; and
a camera controller for identifying the location of the selected hole for the at least one reaction tube.

38. The method of claim 37, wherein the source of ultraviolet radiation comprises at least one light emitting diode having an ultraviolet radiation peak at 308±5 nm, wherein the excitation filter transmits ultraviolet radiation, and blocks visible light between 420 nm and 650 nm, and wherein the imaging filter blocks visible light emitted by the at least one light emitting diode at wavelengths <430 nm.

39. A kit for detecting hop latent viroid, HLVd, nucleic acid infecting a Cannabis plant by RT-LAMP amplification, comprising:
at least one sample collection tube having a cap, a first chosen volume, and containing solid filtration components, comprising: prewashed, activated charcoal, and Chelex Resin in a sample optimization buffer, comprising: Tris-HCl buffer (pH 8), EDTA, Trehalose, BSA (Bovine Serum Albumin), and Tween-20;
at least one fixed-volume micropipette, or at least one fixed-volume capillary tube having a plunger or bulb;
at least one optically clear reaction tube having a second chosen volume, each tube having a cap, and containing a lyophilized reaction pellet, comprising: DNA polymerase, reverse transcriptase, deoxyadenosine triphosphate, deoxycytidine triphosphate, deoxyguanosine triphosphate, deoxythymidine triphosphate, BIP, F3, B3, Loop F, and Loop B first oligonucleotide primers for hybridizing with the HLVd nucleic acid sequence, BIP, F3, B3, Loop F, and Loop B second oligonucleotide primers for hybridizing with the Cannabis sativa gene nucleic acid control, an annealed first FIP primer conjugated to a first fluorophore and having SEQ ID NO: 1, with a first reverse complementary oligonucleotide conjugated to a first quencher for the first fluorophore and having SEQ ID NO: 13, an annealed second FIP primer conjugated with a second fluorophore and having SEQ ID NO: 7, with a second reverse complementary oligonucleotide conjugated to a second quencher for the second fluorophore and having SEQ ID NO: 14, magnesium sulfate, Tris(hydroxymethyl)aminomethane hydrochloride, ammonium sulfate, potassium chloride, and Tween-20;
at least one sample collection tube holder adapted for holding a chosen number of sample collection tubes;
a dry heating block or a water bath capable of being heated, for heating the at least one optically clear reaction tube to about 65° C.;
at least one reaction plate capable of holding at least one reaction tube;
apparatus for exciting and viewing fluorescence from the fluorophores; and
at least one black reaction plate for holding reaction tubes for fluorescence visualization.

40. The kit of claim 39, wherein said apparatus for exciting and viewing fluorescence from the fluorophores, comprises: a source of UV radiation, and a visualization box, having a small viewing hole at the top and fitted with a safe-viewing UV blocking lens for providing a dark environment, for viewing the fluorescence emissions by eye, or for image photographing using a mobile telephone or camera.

41. The kit of claim 39, wherein said apparatus for exciting and viewing fluorescence from the fluorophores, comprises: an image acquisition system, comprising:
a reaction tube holder, having a top surface and a bottom surface parallel thereto, a selected distance therebetween, and a chosen number of holes formed between the top surface and the bottom surface, whereby a portion of the at least one reaction tube having a chosen length and placed in a selected hole extends below the bottom surface when the top of the at least one reaction tube is positioned in the vicinity of the top surface;
a source of ultraviolet radiation for irradiating the portion of the at least one reaction tube extending below the bottom surface;
an excitation filter for selecting a wavelength range of ultraviolet radiation from said source of ultraviolet radiation effective for exciting fluorescence excitation in the at least one fluorophore of the at least one reaction tube;
an imaging filter for blocking ultraviolet radiation and transmitting fluorescence excitation from the at least one fluorophore;
a camera for receiving the transmitted fluorescence excitation from the at least one fluorophore; and
a camera controller for identifying the location of the selected hole for the at least one reaction tube.

42. A kit for detecting hop latent viroid, HLVd, nucleic acid infecting a Cannabis plant by RT-LAMP amplification, comprising:
at least one, screw-capped sample collection tube having a first chosen volume and containing solid filtration components, comprising: prewashed, activated charcoal, and Chelex Resin in a sample optimization buffer, comprising: Tris-HCl buffer (pH 8), EDTA, Trehalose, BSA (Bovine Serum Albumin), and Tween-20; and
at least one optically clear reaction tube having a second chosen volume, each tube having an attached snap cap, and containing a lyophilized reaction pellet, comprising: DNA polymerase, reverse transcriptase, deoxyadenosine triphosphate, deoxycytidine triphosphate, deoxyguanosine triphosphate, deoxythymidine triphosphate, dUTP and thermolabile uracil DNA glycosylase, BIP, F3, B3, Loop F, and Loop B first oligonucleotide primers for hybridizing with the HLVd nucleic acid sequence, BIP, F3, B3, Loop F, and Loop B second oligonucleotide primers for hybridizing with the Cannabis sativa gene nucleic acid control, an annealed first FIP primer conjugated to a first fluorophore and having SEQ ID NO: 1, with a first reverse complementary oligonucleotide conjugated to a first quencher for the first fluorophore and having SEQ ID NO: 13, an annealed second FIP primer conjugated with a second fluorophore and having SEQ ID NO: 7, with a second reverse complementary oligonucleotide conjugated to a second quencher for the second fluorophore and having SEQ ID NO: 14, magnesium sulfate, Tris(hydroxymethyl)aminomethane hydrochloride, ammonium sulfate, potassium chloride, and Tween-20.
